(19) United States
(12) Patent Application Publication
(10) Pub. No.: US 2023/0011945 A1
(43) Pub. Date: Jan. 12, 2023

(54) REVERSIBLE ENDOLUMINAL OCCLUSION DEVICE FOR HEALING OF COLORECTAL ANASTOMOSIS

(71) Applicant: UNIVERSITY OF FLORIDA RESEARCH FOUNDATION, INCORPORATED, Gainesville, FL (US)

(72) Inventors: Chelsey S. SIMMONS, Gainesville, FL (US); Sanda A. TAN, Gainesville, FL (US)

(21) Appl. No.: 17/784,733

(22) PCT Filed: Dec. 14, 2020
(86) PCT No.: PCT/US2020/064885

§ 371 Date: Jun. 13, 2022

Related U.S. Application Data
(60) Provisional application No. 62/947,626, filed on Dec. 13, 2019.

(51) Int. Cl. A61B 17/12; A61M 25/10

(52) U.S. Cl. CPC A61B 17/1204; A61B 17/12099; A61B 17/12136; A61M 25/1011; A61M 2025/1052

(57) ABSTRACT

Disclosed herein are apparatus embodiments and methods for using same that facilitate occlusion of a lumen in a subject. In a particular example, the apparatus is configured for occluding a portion of the lower GI tract (e.g. colon). Further, the apparatus is configured for placement in the lumen and insufflation to secure within the lumen such that the apparatus remains in place without the need for uncomfortable conduits or tools extending out of the patient. In a particular method embodiment, the apparatus is useful for occluding a lumen following a surgical procedure such as an anastomosis.

### BACKGROUND

Each year more than 600,000 surgical procedures are performed in the United States to treat a number of colon and rectum-related diseases, including both benign and malignant diseases such as colorectal cancer, polyps, IBD (inflammatory bowel disease including Crohn's disease and ulcerative colitis), and colitis. These surgical procedures often include resection of the bowel resulting in anastomoses to join portions of the bowel back together following surgery. Passage of enteric content through the bowel during the healing time can cause severe complications to a patient. One method to avoid these severe complications, including sometimes death, and to allow the anastomosis to heal includes diverting enteric content away from the anastomosis, proximal to the connection point. One way in which this is accomplished is by placing an ostomy bag in the patient, such that the ostomy bag receives a piece of bowel proximal to the connection point through the abdominal wall creating another exit for the enteric content to exit the body as a diversion. However, the ostomy is generally a temporary solution, resulting in a second surgery for the patient. Furthermore, an ostomy itself is an inconvenience for patients and their caretakers, and can cause additional complications.

### BRIEF DESCRIPTION OF THE DRAWINGS

A more particular description briefly stated above will be rendered by reference to specific embodiments thereof that are illustrated in the appended drawings. Understanding that these drawings depict only typical embodiments and are not therefore to be considered to be limiting of its scope, the embodiments will be described and explained with additional specificity and detail through the use of the accompanying drawings in which:

FIG. 1 provides a cross-sectional view of a portion of a scope with an embodiment of an apparatus as described herein, and an insufflator tool disposed within the scope, in connection with a portion of the apparatus.

FIG. 2 depicts another embodiment of an apparatus described herein, positioned within a portion of a large bowel of a subject.

FIGS. 3A-3C provides examples of apparatus embodiments described herein placed by endoscope.

FIGS. 4A-4D provide various examples of embodiments of an inflatable body or variations thereof, described herein, including non-limiting rigid regions to generate non-uniform inflated geometries.

FIGS. 5A-5C provide various geometries of the inflatable body, including, for example, multiple stackable, optionally interconnectable, inflatable bodies as shown in FIGS. 6B-6C.

FIGS. 6A-6B provide examples of the apparatus embodiments positioned in subject colon, showing, for example, components of the apparatus which may extend through the rectum during use of the apparatus.

### DETAILED DESCRIPTION

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

It is to be noted that the terms “first,” “second,” and the like as used herein do not denote any order, quantity, or importance, but rather are used to distinguish one element from another. The terms “a” and “an” do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.” The modifier “about” used in connection with a quantity is inclusive of the stated value and has the meaning dictated by the context (e.g., includes the degree of error associated with measurement of the particular quantity). It is to be noted that all ranges disclosed within this specification are inclusive and are independently combinable.

The term “material” as used herein refers to any fluid, solid or semi-solid material that passes through a lumen of a subject. Material may include blood, other bodily fluid, enteric contents, or fecal material.

The term “occlude”, or “occluding” or other grammatical forms thereof, as used herein, refers to a partial or complete blockage. For example, to occlude the lumen means to partially or completely block passage of material through the lumen.

The term “insufflation conduit” as used herein describes a conduit through which fluid (gas and/or liquid) may pass, and/or by which fluid may be delivered, and comprises a tube, a catheter, or the like.

The term “patient” as used herein refers to a human or non-human animal. Non-human animals for which the apparatus embodiments are particularly useful include dogs, cats, cows, horses, pigs and goats.

The terminology used herein is for the purpose of describing particular embodiments only and is not intended to be limiting. As used herein, the singular forms “a,” “an,” and “the” are intended to include the plural forms as well, unless the context clearly indicates otherwise these terms do not denote a limitation of quantity, but rather denote the presence of at least one of the referenced items.

### Overview

Anastomoses serve to surgically connect two separate (severed) luminal structures resulting in a continuous channel. This procedure is often used following colon and rectum surgery, where a patient requires a resection. Following the re-connection of the bowel, the surgical site requires healing time without the passage of stool, which could irritate the site of the anastomosis and could risk contamination of the site of the resection, as well as cause leakage of stool through the anastomosis during healing. Such leakage (i.e. anastomotic leak) could be caused as a result of passage of material, such as stool, through the bowel following surgery due to inflicting tension on the anastomosis, resulting in inadequate blood supply to the anastomosis, or disrupting the staple line, for example. An anastomotic leak may result in stool exiting the bowel and penetrating the peritoneal or pelvic cavity, causing peritonitis and potentially death to the patient.

Existing post-colon and rectum resection standard of care involves diversion of the flow of stool from the bowel, such that a port is made to allow flow of material from the stomach out of the body via an ostomy bag. This requires a first additional surgical procedure to create the opening in the abdominal wall to connect the ostomy, and later, a second additional surgical procedure to remove the ostomy and close the port in the abdominal wall once the anastomosis is fully healed and the stool can flow through the bowel again without risk to the surgical site. Surgical procedures carry medical risks, and often a high financial burden. Having multiple surgical procedures following a bowel resection or other procedure requiring anastomosis, in order to place an ostomy requires downtime for the patient, risks associated with the placement of the opening, risks associated with the removal of the ostomy and healing from the multiple surgeries involved, in addition to the financial burdens.

It has been identified herein, embodiments of an apparatus and a method for temporarily occluding a bowel to allow healing of at least a portion thereof. In some instances, the embodiments described herein may be used for post-surgical treatment of a patient without the need for an ostomy until the surgical site heals. Embodiments of an apparatus for placement in a lumen (e.g., colon or rectum) of a patient are provided herein, wherein the apparatus serves to prevent the flow of fluid in the lumen at the placement site. Consequently, post-surgical resection in the colon or rectum, for example, or for other indications some of which are described herein requiring diversion of material from a certain part of the colon to allow healing, including for example, in addition to post-anastomosis following a colectomy, the apparatus and method embodiments may be used for temporary incontinence caused by trauma, for example, to colorectal nerves, or following mild to moderate trauma, for example, in the case of object insertion, or following puncture or other breach of bowel wall during colonoscope or other outpatient procedure, for example, during a polyp removal.

In one embodiment, an embodiment of the apparatus may be placed proximal to the anastomosis to obstruct the bowl and prevent stool from traversing the site of the anastomoses to prevent irritation, maintain sterilization of the surgical site, and promote healing at the site. The apparatus may be temporarily placed until healing of the anastomotic site is complete. The apparatus may be placed and removed by way of endoscopic tools known in the art, some of which are described herein. The apparatus and method embodiments eliminate the need for an ostomy bag to divert passage of material away from the surgical site. Therefore, the apparatus and methods described herein reduce healing time from surgical procedures that are required to both place and remove an ostomy bag.

In one embodiment, an apparatus for obstructing a lumen of a patient is provided, including a removable, inflatable body defining a cavity. The body is provided for placement in a lumen of the subject, to obstruct the lumen occluding the lumen, and in some non-limiting cases, forming a seal between the body and the lumen at a point or points of placement of the inflatable body to at least partially block the passage of material through the lumen at the point or points of placement.

As described herein, the inflatable body can be inserted into the lumen, in some instances in a pre-inflated state, and once placed at the point of placement, the apparatus can be inflated to expand the body such that it interfaces with the walls of the lumen within which it is placed. In some non-limiting examples, the inflatable body may be fully deflated during insertion, placement, and removal of the apparatus. In other non-limiting examples, the inflatable body may not be fully deflated during insertion, placement and removal of the apparatus. This inflation of the body occurs, in some examples, by delivery of fluid to the cavity of the body such that the body can form a seal with the lumen to temporarily occlude the fecal stream.

In some non-limiting examples, components of the apparatus may be made of any biocompatible material suitable for implantation into a mammalian body or combinations thereof. Different portions of the apparatus may or may not be made from different materials or combinations of materials. For example, portions of the apparatus such as the inflatable body may be formed of an elastic polymer, for example silicone or polyurethane (e.g., pellethane). Portions of the apparatus may also include a flexible polymer. Components of the apparatus may be radiopaque or include discrete radiopaque markers, such as within the balloon wall, along the insufflation conduit or at or near the insufflation conduit tip, in some non-limiting examples. The apparatus components may include a textured portion or textured surface to reduce or eliminate slippage or movement of the apparatus along the intestinal wall. In some non-limiting examples, the inflatable body may be formed of a textured material on a micron or millimeter scale to reduce or eliminate movement of the inflatable body once placed or once inflated within the lumen. In some embodiments the materials incorporated may be sterilized prior to use. Appropriate sterilization techniques can be adapted based on recommended techniques for the materials used in the apparatus. In some embodiments a drug, such as an antibiotic, may be incorporated into the apparatus, as an extra precaution or means of treating the area around the anastomosis. The apparatus may also be used in conjunction with a sealant or sclerosing solution which may be used to seal or glue the device to the bowel wall. Several possible sealants or glues are commercially available.

### Description of Illustrated Embodiments

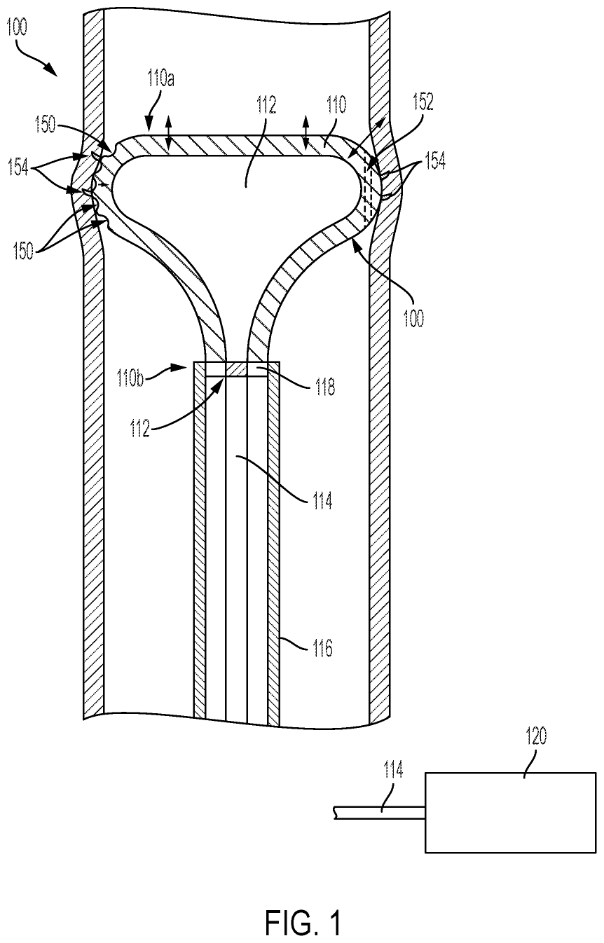

Turning to the drawings, FIG. 1 provides an embodiment of an apparatus 100 for obstructing a lumen of a patient. The apparatus 100 includes an inflatable body 110 for placement in a lumen of a subject to occlude the lumen at a point of placement. The inflatable body comprising a first end 110a and a second end 110b, the second end 110b comprising a port 112 for removably engaging an insufflation conduit 114 to inflate the inflatable body 110 to an inflated state. Once the body 110 has reached a desired inflated state, the insufflation conduit 114 may be disengaged from the port 112 thereby leaving the body 110 to seal the lumen from passage of material, in one non-limiting embodiment. In another non-limiting embodiment, the insufflation conduit 114 may be connected to the second end 110b of the device. The connection may be a direct connection, or may be an indirect connection by way of a connector component which may be rigid or flexible, in one non-limiting embodiment. A valve may be provided in the fluid flow pathway to selectively allow or prevent fluid from entering or exiting the apparatus 100. In one embodiment, the valve may be disposed in the fluid flow pathway at or near the connection point between the conduit 114 and the body 110 (which in some instances may include the port 112). The valve may allow fluid to enter the cavity from the insufflation conduit 114 and may prevent fluid from escaping the apparatus 100 until released. The valve may include a one-way valve, in one non-limiting embodiment. Following inflation, the insufflation conduit 114 may either be removed from the device, or the insufflator conduit 114 may be severed, wherein a portion of the conduit may be left in the body. In another example, the insufflation conduit 114 may be allowed to remain associated with the apparatus 100 until the apparatus is removed from the patient. In one example, the apparatus 100 may be removable from or insertable into the lumen in its deflated or partially inflated state. In a further, non-limiting embodiment, the inflatable body 110 may be fully inflated prior to insertion and placement of the body 110 within the lumen of the patient. In one example, when the body 110 is in the deflated state, a width of the second end 110b may include a range of 24-34 mm.

To deflate the apparatus 100, an object such as a sharp or blunted needle or a wire may be inserted via scope to traverse or puncture the valve to allow air to escape the cavity. Once the cavity is sufficiently depleted of air, the object may be removed along with or separately from the device from the lumen. In another embodiment, fluid may be allowed to escape the apparatus over time. In yet another embodiment, the fluid may be withdrawn from the apparatus. Typically, the apparatus 100 would remain inflated in the lumen for a number of days to allow the surgical site to heal prior to deflation and removal of the apparatus 100 from the lumen.

Other connection mechanisms may be used at the insufflation conduit 114 and apparatus 100 interface, including a luer-lock connection or a quick release valve, in non-limiting embodiments. Connection mechanisms may further be used to connect one inflatable body to another inflatable body, or to connect an inflatable body to an insufflation conduit, for example.

In one embodiment, the apparatus 100 may further include an insufflation conduit 114 for affixing to the port 112 of the apparatus 100. The insufflation conduit 114 may be used for delivering a fluid to the inflatable body 110 to expand the inflatable body 110, in one embodiment, or a portion of the apparatus 100 from the deflated state to the inflated state. In order to deliver fluid to the apparatus 100, in one embodiment, at least one end of the insufflation conduit 114 must connect to the apparatus 100, as described above. At least another end of the insufflation conduit 114 may be associated with an insufflator 120 for delivering a fluid to the apparatus 100. The fluid being delivered may include a gas or a liquid, wherein upon delivery of the fluid to the apparatus 100, the body 110 may expand to the dimensions of the lumen in which it is placed. In other embodiments, the apparatus 100 may expand beyond the dimensions of the lumen in which it is placed. In at least one embodiment, the body 110 may include a flexible material.

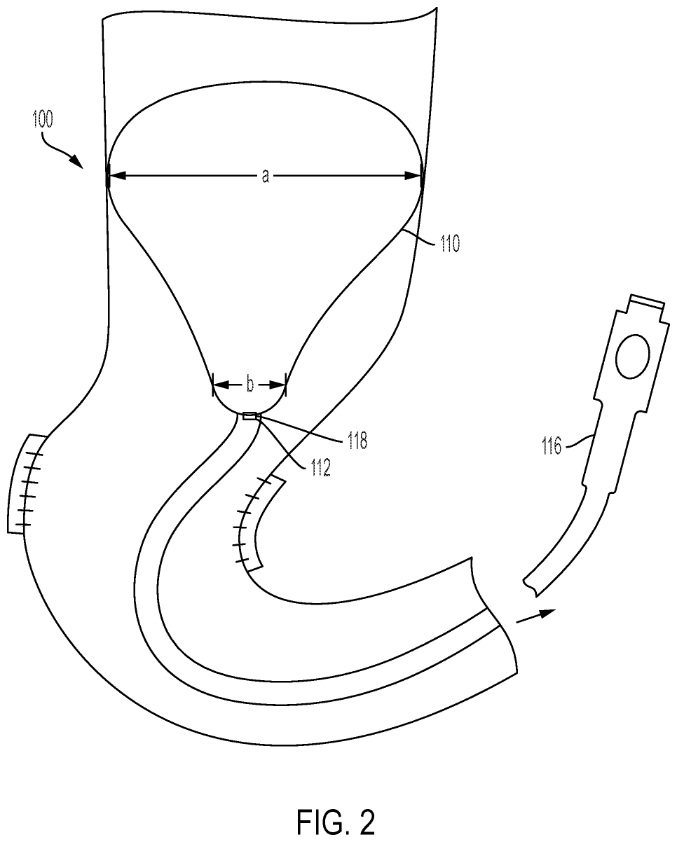

In its inflated state, the first end 110a of the body may include a first diameter “a” and the second end 110b may include a second diameter “b” as shown, for example in FIG. 2, wherein the body 110 tapers down from the first diameter “a” to the second diameter “b”. The body 110 therefore may expand to conform to the lumen in which it is placed to form a seal within the lumen. Inflation of the body 110 may cause the first end 110a to expand to a larger profile than the second end 110b, in one non limiting embodiment. In one non-limiting embodiment, the first diameter “a” may range from 15-65 mm, and the second diameter “b” may range from 0.5-49 mm.

Inflation of the body 110 may also cause vertical elongation of the device 100 in some examples. The body 110 may be configured to associate with a placement tool 116. The placement tool 116 may be used to direct and place the device 100 in the lumen, with or without the use of imaging for correct placement. In at least one embodiment, the placement tool 116 may be used to place the device 100 at the target position near the anastomoses in the colon or rectum and may then be removed from the subject. In one example, the placement tool 116 may include a scope 116 (shown in FIG. 1). In a further example, the scope may include a colonoscope or an endoscope in non-limiting embodiments. The apparatus 100 may be placed onto a portion of the scope for introduction of the apparatus 100 into the body. In one example, a colonoscope which has an additional channel for receiving the apparatus, and placement of the apparatus in the correct location in the lumen, may be used. Once the apparatus 100 is located to the site of the anastomoses by way of the scope 116, the apparatus 100 may be inflated, with or without visualization by way of an imaging device during deployment of the apparatus 100. In one non-limiting embodiment, the scope 116 may associate with the apparatus 100 via a rigid portion 118 of the body at the second end 110b.

The placement of the apparatus 100 may occur during the initial colorectal surgery and would not necessitate a separate surgical procedure. However, if needed, the placement of the apparatus 100 could occur independent of the initial colorectal surgery. In some embodiments, the apparatus 100 may be placed to occlude the lumen prior to the surgical procedure. In other examples, the apparatus 100 may be placed in the lumen during, or following the surgical procedure.

Examples of placement tools including scopes 116 have been described herein for manipulation and placement of the apparatus 100 inside and outside of the subject; however, in some examples, the apparatus 100 may be placed by way of other mechanical tools, including but not limited to pliers, or a clamp-type hand tool, particularly in cases in which the apparatus 100 is being placed in the rectum for example.

The apparatus 100 may, as described herein, be used to seal a portion of a lumen, such as for example, a colon, or a rectum or other luminal structure prior to, during, or following a surgical event. In one embodiment, the surgical event may include an anastomosis resulting from a resection or other colorectal, or other procedure. The apparatus 100 described herein in embodiments may be removable. For example, the apparatus 100 may be placed into the lumen for a short duration to allow for healing of a surgical site downstream of the apparatus 100. The apparatus 100 may be left in the lumen without any connection to the environment outside of the subject for the duration. Following a period of time, the apparatus 100 may be removed from the subject by the use of placement tools 116 described herein, including, for example an endoscope, a colonoscope, pliers, or other similar devices.

In some non-limiting embodiments, at least a portion of the apparatus body 110 may include optional features to provide drainage. In one embodiment, these features may allow fluid to bypass the apparatus 100. In another embodiment, these features may allow fluid to drain through a portion of the apparatus 100. In one example the feature may include an external porous region 150 to allow fluid to bypass the apparatus 100. This structure may be provided in an outer wall of the body 110 as shown in FIG. 1, for example. In another non-limiting embodiment, the apparatus 100 may include one or more channels 152 to allow fluid to pass through a wall of the body 110 as shown in FIG. 1. The channels 152 may be provided as an alternative to, or in addition to the porous region(s) to provide drainage or fluid flow in non-limiting embodiments. In another embodiment, one or more protrusions 154 may be provided extending from the apparatus 100 to position the apparatus body 110 more effectively by allowing the protrusion(s) to embed in the wall of the lumen. Consequently, in at least one non-limiting embodiment, the protrusion 154 may include one or more sharp features for gripping onto or embedding into the wall of the lumen for placement and securing the apparatus body 110 therein.

In one example, therefore, a method for obstructing a lumen in a subject to allow healing of a surgical site in the lumen is provided. The method includes insertion of an apparatus 100 into the lumen with a placement tool 116. The apparatus 100 is placed upstream of, or proximal to the surgical site or the intended location of the surgical site, or proximal to the target point, which could include a point of injury, or other as described herein, and placement of the apparatus 100 may occur while the apparatus 100 is in a deflated position. Upon reaching the point of placement, the apparatus 100 may be inflated. In this manner, an insufflator conduit 114 may be connected to a port of the apparatus 100 to deliver fluid to the apparatus 100 until the apparatus 100 reaches the required inflated state to form a seal or partial seal between the body and the lumen at least partially blocking fluid or material from bypassing the apparatus 100 at least in the region of the surgical site or anastomoses. Thereafter, the placement tool 116 is removed from the conduit leaving the apparatus 100 in the lumen. Following a duration of time to allow healing of the surgical site, the placement tool 116 may be reinserted into the conduit and attached to the apparatus 100 to remove the apparatus 100 therefrom, in one non-limiting embodiment.

In another non-limiting embodiment, a larger device (10-50 mm diameter, for example) is positioned at the end of a multi-lumen scope and a tether may be fed through one lumen of the scope before it is inserted into a subject. By holding the tether at the back end of the scope, the apparatus 100 can be pushed into place by the scope as it is inserted in the bowel and then inflated.

In one embodiment, a kit may be provided including an apparatus 100 having an inflatable body 110. The apparatus for placement in a lumen of a subject to form a seal in the lumen at a point of placement, at least partially blocking the passage of fluid or material through the lumen. The kit may include a placement tool 116, an insufflator conduit 114 for connecting to an insufflator 120 for providing fluid to expand the apparatus from a deflated state to an inflated state. The fluid flow may be controlled by way of a valve in the fluid flow pathway. The valve may include a one-way valve, in one non-limiting embodiment. In some embodiments, the insufflator, or other device may connect to the apparatus 100 to deflate the apparatus from the inflated state to a deflated state for removal of the apparatus 100 from the subject. The insufflator conduit may connect to the apparatus in a number of ways. In one example, a rigid portion may be removably, or permanently affixed to the apparatus 100, and may connect to the insufflator conduit for providing fluid to the apparatus 100.

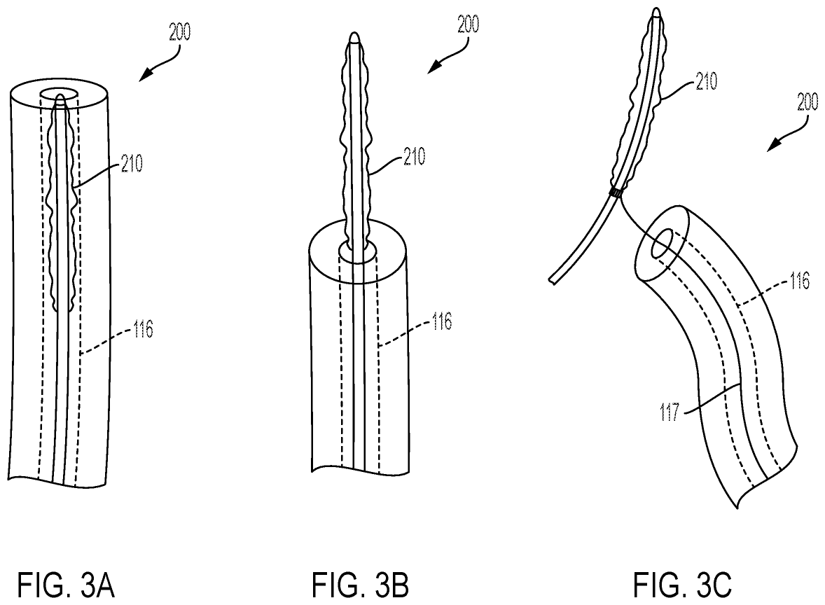

FIGS. 3A-3C provides examples of apparatus embodiments 200 described herein placed in the subject by an endoscope 116. In the embodiments shown in FIGS. 3A-3C, the inflatable body 210 is shown in a deflated state. The endoscope 116 may be used to position the inflatable body 210 in the lumen prior to inflating the inflatable body 210, in a non-limiting embodiment. A guidewire 117 may be used to position the inflatable body 210. The guidewire 117 is shown inside the lumen of the endoscope 116 in FIG. 3C. While the size of the lumen within the endoscope may vary, in at least one example, such as in the embodiment shown in FIG. 3C, wherein the inflatable body 210 is positioned outside the scope lumen and only the guide wire 117 is positioned within the scope 116, the diameter of the lumen may be between 0.3 mm-6 mm. In the example shown in FIG. 3A the inflatable body 210 is positioned within the endoscope 116 lumen. In this instance, the lumen may be at least 6 mm or larger, to accommodate the inflatable body 210, in one non-limiting example. FIG. 3B provides an example wherein the inflatable body 210 may be partially extending form the endoscope 116 during use; however, the insufflation conduit may be maintained within the scope 116 as shown in the Figure. In this particular non-limiting example, the lumen may be of moderate size and may include a diameter of between 2 mm-6.5 mm. In some examples, for example, in embodiments shown in FIGS. 3B-3C, the inflatable body 210 may be placed into the lumen by hand prior to the anastomosis. Following anastomosis the connection to the inflatable body 210 may be made to inflate the inflatable body 210 following anastomosis. When the inflatable body is placed within the lumen prior to anastomosis, following the anastomoses, the interconnecting components used to inflate the inflatable body 210 may be introduced by endoscope and connected thereto.

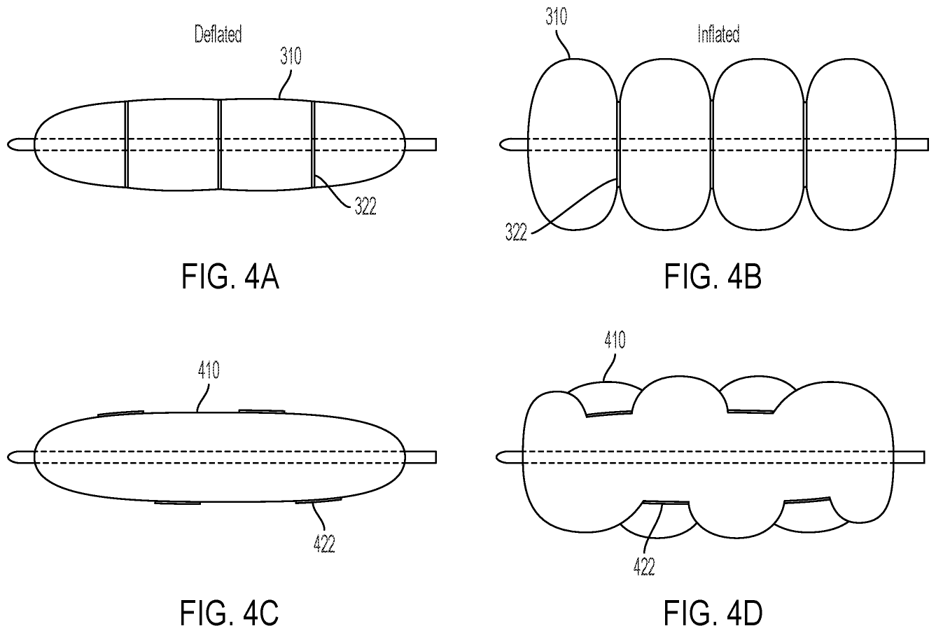

FIGS. 4A-4D provide various examples of embodiments of an inflatable body providing variations thereof, including non-limiting rigid regions to generate uniform or non-uniform inflated geometries. For example, in FIGS. 4A-4B is provided an embodiment of an inflatable body 310, wherein one or more rigid portions or rigid rings 322 are associated with or positioned on or around the inflatable body 310 as shown, such that when inflated, the inflatable body 310 changes from a deflated state as shown in FIG. 4A to an inflated state as shown in FIG. 4B, wherein the inflatable body 310 is constricted where the rigid rings or portions 322 are positioned. This enables control of and the ability to adjust the geometry of the inflatable body 310. In another example shown in FIGS. 4C-4D, is an inflatable body 410 with rigid components 422 positioned along its surface, wherein the rigid components 422 serve to deflect portions or areas of the inflatable body 410 as the inflatable body 410 is expanded by inflation. As shown in the deflated state in FIG. 4C, the inflatable body 410 can be expanded to, for example, the state shown in FIG. 4D wherein the rigid components 422 provide points of deflection of the inflatable body 410 changing its geometry.

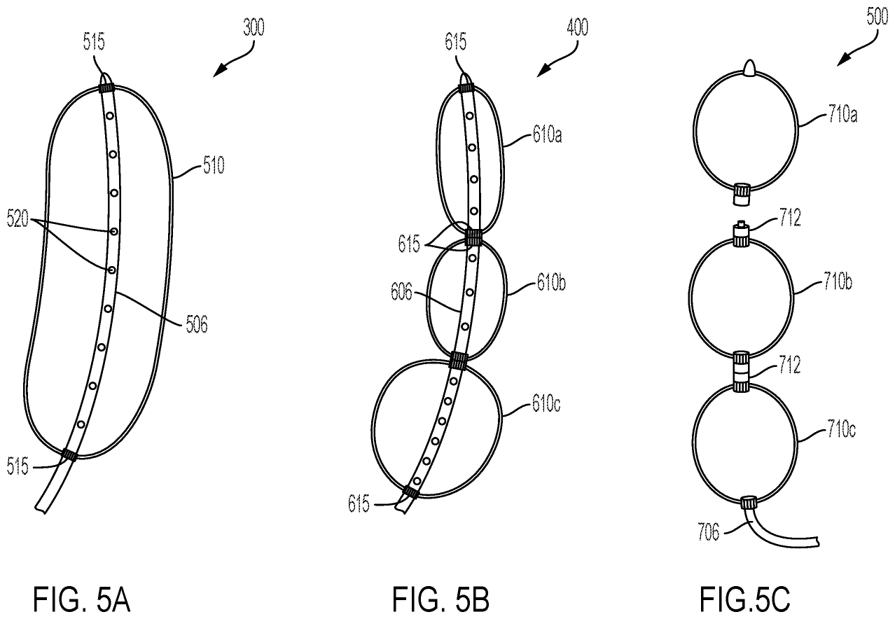

FIGS. 5A-5C provide various geometries of the inflatable body, including, for example, multiple stackable, optionally interconnectable, inflatable bodies as shown in FIGS. 5B-5C. FIG. 5A provides a cross-sectional view of an embodiment 300 of the apparatus, wherein an inflatable body 510 is shown associated with an insufflation conduit 506. The inflatable body 510 is connected to the insufflation conduit 506 at two points along the insufflation conduit 506 by a securing member 515. The insufflation conduit 506 includes a plurality of inflation ports 520 along its length, which deliver a flow of fluid from the insufflation conduit 506 into the inflatable body 510.

FIG. 5B provides another embodiment 400 of the apparatus, comprising multiple inflatable bodies 610a, 610b, 610c disposed, end to end, along the length of an insufflation conduit606. The insufflation conduit 606, as in the embodiment shown in FIG. 5A, includes a plurality of inflation ports 620 provided to deliver fluid to the multiple inflatable bodies 610a, 610b, 610c. Each individual inflatable body 610a, 610b, 610c is attached to the insufflation conduit 606 by way of securing members 615 both at the first end and second end of each inflatable body 610a, 610b, 610c, for example. In this manner, fluid (e.g. gas, liquid) may be directed through the insufflation conduit 606 and delivered to each inflatable body 610c610b, 610a via the insufflation conduit inflation ports 615.

In yet another embodiment 500 shown in FIG. 5C, multiple inflatable bodies 710 are connected end-to end. This connection may occur via connection ports 712, in one example, which allow the flow of fluid to pass from one inflatable body 710a, 710b, 710c to an adjacent inflatable body 710a, 710b, 710c. In this particular, non-limiting embodiment, only one inflatable body 710c is associated with the insufflation conduit 706 or other fluid delivery device. Fluid is delivered to the first inflatable body 710c, and via the connection ports 712, fluid is delivered to each inflatable body 710b, 710a, in the sequence. Fluid is delivered to inflate each inflatable body to an inflated state wherein in the inflated state, a contact may be formed between one or more of the multiple inflatable bodies 710 and the inside of a lumen of a subject to obstruct the lumen at the point at which contact between the lumen and the apparatus, particularly, between the inflated inflatable body and the lumen, occurs. In other examples, the connection of the inflatable bodies to one another may occur via other approaches. For example, the individual inflatable bodies may be integrally connected to one another, or connected through use of an adhesive (e.g., glue). Further, the inflatable bodies described in various embodiments herein may be anchored to the insufflation conduit by a variety of means, including, for example, by glue or other adhesive, by a securing member or connection ports described herein, or a combination thereof.

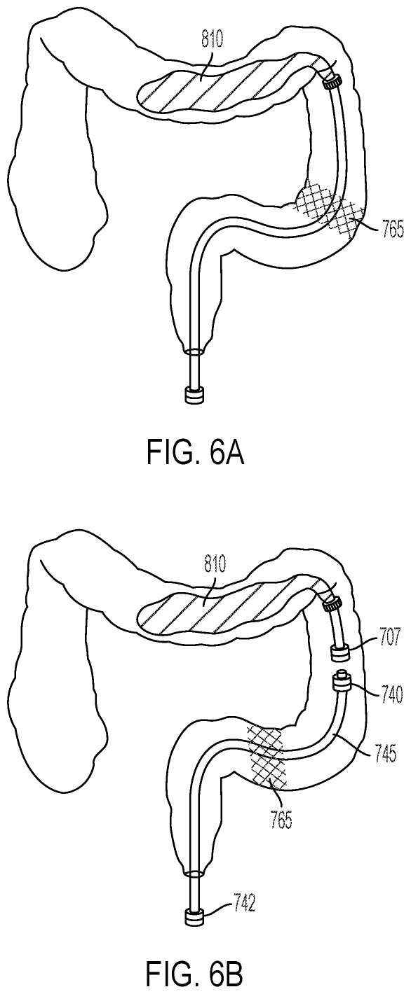

FIGS. 6A-6B provide examples of the apparatus embodiments positioned in a subject colon, showing, for example, components of the apparatus which may extend through the rectum during use of the apparatus. It is noted that reference to the inflatable body 810 in FIGS. 6A-B is provided for discussion purposes only and is intended to be representative of the inflatable body embodiments described herein. In some examples, an inflation port may be accessible external to the subject's body (i.e., via the rectum) as shown in FIG. 6A. FIG. 6B shows another embodiment in which an endoscope (not shown in FIG. 6A-6B) can be used to place the inflation port in the lumen of the subject. In this case, the juncture between an insufflation port 707 and a first end of a connector (inflation port 740) to join to the inflator is positioned within the lumen of the subject. The second end 742 of the connector 745 may be connectable to the inflator (not shown in FIG. 6A-6B). outside the body of the subject as shown, for example. In other examples, the inflation port may be positioned in the subject's body, and a connector may be inserted into the body to connect to the inflation port within the subject.

As depicted in FIGS. 6A-6B, the injured region or anastomoses 765 of the subject is indicated, and the inflatable body and connectors are positioned proximal to the injured region or anastomoses 765. The apparatus may be positioned prior to a surgical event, in some embodiments. In other examples, the apparatus may be positioned in the lumen following an injury or surgical event to eliminate or significantly reduce the passage of material through the lumen at the point of contact between the apparatus and the lumen.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims

What is claimed is:

1. An apparatus for occluding a lumen of a patient, comprising:
an inflatable body for placement in a lumen of the patient, the inflatable body comprising a first end and a second end, the second end comprising a port for removably engaging an insufflation conduit to inflate the inflatable body to an inflated state, such that when the body is in the inflated state, the conduit may be disengaged from the port allowing the inflatable body to remain thereby occluding the lumen at the location of placement.

2. The apparatus of claim 1, wherein the apparatus is removable from or insertable into the lumen when the inflatable body is in a deflated state.

3. The apparatus of claim 1, further comprising an insufflation conduit for affixing to the port, said insufflation conduit for delivering a fluid to the inflatable body to expand the inflatable body to the inflated state.

4. The apparatus of claim 1, wherein the insufflation conduit is associated with an insufflator for delivering a fluid.

5. The apparatus of claim 4, wherein the fluid is a gas or a liquid.

6. The apparatus of claim 1, wherein the body comprises a flexible material.

7. The apparatus of claim any of claims 1-5, wherein, in the inflated state, a first portion of the inflatable body comprises a first diameter and a second portion of the inflatable body comprises a second diameter, wherein the body tapers down from the first diameter to the second diameter.

8. The apparatus of any of claims 1-5, wherein inflation of the body causes the first portion to expand to a larger profile than the second portion.

9. The apparatus of any of claims 1-8, wherein the body is configured to associate with a scope for placement in the lumen of the subject.

10. The apparatus of claim 9, wherein the scope associates with a rigid portion of the body at the second end.

11. The apparatus of claim 1, wherein the surgical event comprises an anastomosis.

12. The apparatus of claim 1, wherein the apparatus is removable.

13. The apparatus of any of claims 1-12, wherein a width of the second end is of a range of 24-34 mm when the body is in a deflated state.

14. The apparatus of claim 1, further comprising an insufflation conduit comprising an inflation port, wherein the inflatable body is associated with the insufflation conduit, said insufflation conduit delivering fluid to the inflatable body through the inflation port to expand the body to an inflated state.

15. The apparatus of claim 14, further comprising multiple inflatable bodies associated end to end, wherein at least one of the multiple inflatable bodies is associated with the insufflation conduit, such that fluid flow passes from the insufflation conduit through the multiple inflatable bodies to expand the bodies to an inflated state.

16. The apparatus of claim 14, further comprising multiple inflatable bodies associated with the insufflation conduit, wherein said insufflation conduit comprises multiple inflation ports to deliver a fluid to the multiple inflatable bodies to expand the inflatable bodies to an inflated state.

17. The apparatus of claim 15, wherein each of the multiple inflatable bodies comprise a first end and a second end, said first end of one of the multiple inflatable bodies may be connected to the second end of another of the multiple inflatable bodies.

18. The apparatus of claim 17, wherein the multiple inflatable bodies may be connectable to one another via their ends by a connection mechanism.

19. The apparatus of claim 18, wherein the connection mechanism comprises a luer-lock system.

20. The apparatus of claim 1, further comprising one or more limiting members disposed on a portion of the inflatable body, such that upon inflation of the inflatable body, the limiting members cause deflection of the portion of the inflatable body associated therewith upon insufflation.

21. A method for occluding a lumen in a subject, comprising:
insertion of the apparatus of claim 1 into the lumen with a placement tool;
placement of the apparatus of claim 1 in the lumen upstream of a target area;
delivering a fluid to the apparatus via a port on the apparatus to inflate the apparatus, such that the apparatus contacts the lumen to occlude the lumen at the point of placement of the apparatus in the lumen;
removal of the placement tool from the lumen, thereby leaving the apparatus in the lumen.

22. The method of claim 21, wherein the delivery of fluid occurs by connecting an insufflation conduit to a port of the apparatus.

23. The method of claim 21, wherein the placement tool comprises a colonoscope or an endoscope.

24. The method of claim 23, w herein the colonoscope comprises at least one channel for receiving the apparatus.

25. The method of claim 21, further comprising visualizing the placement of the apparatus and/or removal of the placement tool with an imaging device.

26. The method of claim 21, further comprising wherein the apparatus is in a deflated position during the placement step.

27. A kit comprising:
an apparatus comprising one or more inflatable bodies, said bodies for placement in a lumen of a subject at a point of placement upstream of a target area, said one or more bodies comprising a port for receiving a fluid;
a placement tool;
an insufflation conduit for delivering fluid; and optionally, an insufflator for providing fluid to the apparatus to expand the body to an inflated state.

28. The kit of claim 27, wherein the insufflation conduit comprises one or more inflation ports for delivering fluid to the one or more inflatable bodies.

29. The kit of claim 27, further comprising one or more connection mechanisms for connecting the inflatable bodies to the insufflation conduit and/or connecting the inflatable bodies to one another.

30. The apparatus of claim 1, further comprising one or more pores on the body to provide passage of fluid thereby.

31. The apparatus of claim 1, wherein the body comprises one or more channels to provide a passage of fluid therethrough.

32. The apparatus of claim 1, wherein the body comprises one or more protrusions on an exterior portion thereof to interface with a lumen to secure the apparatus in the lumen.

33. An apparatus for obstructing a lumen of a patient, comprising:
one or more interconnectable inflatable bodies for placement in a lumen of a subject to occlude the lumen at the point of contact between an inflatable body and the lumen;
each inflatable body comprising a first end and a second end, and connectable to one another via a connecting component;
a port positioned on at least one of the one or more interconnectable inflatable bodies for removably engaging an insufflation conduit, said insufflation conduit for inflating one or more of the interconnectable inflatable bodies, wherein when at least one of the inflatable bodies is inflated, the lumen is occluded at the point of contact between the at least one inflatable body and the lumen.
